(19) United States
(12) Patent Application Publication
(10) Pub. No.: US 2018/0014970 A1
(43) Pub. Date: Jan. 18, 2018

(54) CATHETER, SYSTEM FOR TARGET TEMPERATURE MANAGEMENT AND METHOD FOR TARGET BODY TEMPERATURE MANAGEMENT

(71) Applicants: Bernardo Lembo Conde de Paiva, Campo Belo (BR); Raphael Einsfeld Simões Ferreira, Vila Mariana (BR)

(72) Inventors: Bernardo Lembo Conde de Paiva, Campo Belo (BR); Raphael Einsfeld Simões Ferreira, Vila Mariana (BR)

(21) Appl. No.: 15/546,161

(22) PCT Filed: Jan. 27, 2016
(86) PCT No.: PCT/BR2016/050014

§ 371 Date: Jul. 25, 2017

(30) Foreign Application Priority Data
Jan. 28, 2015 (BR) BR102015002008-2

(51) Int. Cl. A61F 7/12; A61F 7/00; A61M 25/00

(52) U.S. Cl. CPC A61F 7/12; A61M 25/0043; A61F 7/0085; A61F 2007/0006; A61F 2007/0069

(57) ABSTRACT

A catheter for target body temperature management, a system for target temperature management, and a method for target body temperature management, including a catheter, system or method for target body temperature management in cavities, such as the nasopharynx route, of a body.

### TECHNICAL FIELD OF THE INVENTION

The present invention describes a catheter for target body temperature management. Furthermore, it also presents a system for target temperature management and a method thereof. The present invention is in the technical fields of Medicine and Engineering.

### BACKGROUND OF THE INVENTION

Brain injury are serious causes of motor and/or cognitive sequels for the subject and, many times, leads the subject to death. The initial brain injuries may become worse in the first 120 hours, mainly in the first 48 hours. In order to avoid progression of brain injuries, some means may be employed in order to reduce cell metabolism aiming cell protection and regeneration.

Therapeutic hypothermia (TH) is considered, at the present, the most effective neuroprotective measure. The application of TH is indicated by the guidelines of the American Heart Association (AHA)¹ and by the systematic reviews of The Cochrane Collaboration² for patients who remain comatose after the reversal of cardiopulmonary arrest (CPA).

Medical conditions such as stroke³ and traumatic brain injury (TBI)⁴ may also benefit from this therapy. The most promising data comes from two recent TBI systematic reviews which pose TH as a suitable treatment for this severe group of neurological patients^{4, 5}.

TH was initially induced by methods which decreased the temperature of the entire body, the so called systemic cooling methods. The reduction of body temperature may lead to brain protection, but can produce severe systemic complications such as systemic hemodynamic disturbances, infections and blood clotting disorders⁶⁻⁹; therefore, the therapeutic cooling should ideally occur strictly to the brain (exclusive brain cooling).

To avoid the deleterious effects of whole body cooling, techniques to selectively cool the brain have previously been investigated¹⁰⁻¹³. Among the most promising for using in humans, those that induce cooling of nasopharyngeal surface enable effective brain hypothermia^{10-12, 14} and are considered as non-invasive because they are minimally invasive, easy to apply and promote fast brain cooling. To date, none of the nasopharyngeal methods was able to achieve the exclusive brain cooling. Despite its effectiveness in promoting reduction of brain temperature, some decrease in body temperature of up to 2.5 degrees Celsius¹⁵ are still observed, which can still cause deleterious systemic side effects. The known intranasal devices have not been designed for prolonged use and novel devices should address this gap.

By preventing the reduction of systemic temperatures, severe and undesirable complications could be avoided¹⁶. It is known that slight body temperature reductions, as little as 1-3° C., are sufficient to produce side effects⁶⁻⁹. Hemodynamic complications are extremely common¹⁷ and are related to disturbances of baroreceptors^{18, 19}, myocardial contractility²⁰⁻²², or secondary to electrolytes changes^{23, 24}. Infections and coagulation disorders can occur through several mechanisms⁶⁻⁹ and worsen the condition of patients.

So far, we have not found studies concerning the effects on systemic and brain physiological mechanisms resulting from selective brain cooling¹⁶. Yet, human studies with nasopharyngeal cooling using evaporation of perfluorocarbon gas presented damage to the nasopharyngeal mucosa resulting from cooling. Adverse events occurred in 16% of cases, such as nasal discoloration (11%), epistaxis (2%) and cold-induced tissue injury (1%)^{25, 26}. The extremely low and uncontrolled temperature of the evaporated gas was probably the cause for the adverse events.

Search in the state of the art in patent and non-patent literature revealed the following documents:

Covaciu et al. 2008¹⁰ discloses selectively cooling of the brain with cold saline circulating inside thin walled balloon catheters presenting an inlet and an outlet port configuration (William Cook Europe, Bjaeverskov, Denmark) introduced into the nasal cavity of pigs. Catheters were coupled to a circuit in which cold saline was circulated by means of a roller pump and cooled with a heat-exchanger machine. Subsequently it was connected to a circuit including a Stockert twin pump and a heat exchanger machine Stockert HCU (heat-cooling unit). Said document presents a balloon device which is restrictedly positioned at the nasal cavity, thereby resulting in a reduced contact of the balloon which limits heat exchange purposes. The inlet and outlet port are adjacently configured which reduces the potential flow to the device limited by the radius of nostrils. For heat exchange purposes, the lower the flow the worse will be the heat exchange.

Busch et al. 2010²⁵ is a safety and feasibility study regarding an evaporative method for nasopharyngeal cooling. The method comprises cooling assemblies with elongate tubular members, a reservoir containing pressurized gas and a manifold connecting the reservoir and the elongate tubular members. The elongate tubular members were inserted only into the patient's nostrils and a pressurized gas was directly delivered onto the surface of the nasal cavity through ports. The method described in said document has no control of the gas temperature vaporized onto the nasal cavities nor continuous feedback control from the brain temperature. Also, the method described in said document promotes direct contact of the evaporated gas with nasal mucosae which may cause adverse events such as periorbital gas emphysema, coolant in facial sinus, epistaxis and tissue damage, as described by the authors. Additionally, the gas may be absorbed by lung circulation.

Doll et al. 2010²⁷ discloses one approach of pharyngeal selective brain cooling (pSBC) in rats. Said document presents a method which includes a roller pump (1.) circulating ice water (2.) through a cooling tube (3.), which is placed in the pharynx of the animal. The inlet and outlet port are adjacently configured which reduces the potential flow to the device limited by the radius of nostrils. For heat exchange purposes, the lower the flow the worse will be the heat exchange. Furthermore, the cooling tube is not expandable, thereby resulting in a reduced contact of the tube with the conformation of the body part which limits heat exchange as well.

US2010217361A1 discloses esophageal heat transfer device which has input port connected with external supply tube and receiving coolant from chiller, output port connected with external return tube, and end cap affixed with coolant supply tube. US2012265172A1 discloses esophageal heat transfer device for inducing hypothermia during surgical procedures to treat cardiac arrest which has proximal end including input port and output port, and distal end configured for insertion into esophagus of patient. US2013006336A1 discloses esophageal heat transfer device, useful e.g. for controlling core body temperature, comprises lumens, distal end for insertion into pharyngeal opening, heat transfer region, heat transfer medium input and output port, and gastric tube. US2014155965A1 discloses a system for controlling core body temperature of patient, which has microprocessor which is coupled to external source and is provided to regulate flow of heat transfer means to the esophagus. All above related devices failed to provide a selective and mainly exclusive body part cooling which leads to known severe adverse events. Furthermore, the methods and systems stated above are restricted to cooling techniques. Depending on the therapeutic purpose, heating can be a suitable treatment choice. Another restriction of the methods and systems stated above is that it is restricted to esophageal cooling. Another restriction of the methods and systems stated above is that the inlet and outlet ports are positioned through the same orifice which reduces the potential flow to the device limited by the radius of the catheter. For heat exchange purposes, the lower the flow the worse will be the heat exchange. Furthermore, the cooling tube used in the systems stated above is not expandable, thereby resulting in a reduced contact of the tube with the conformation of the body part which limits heat exchange as well.

Document US2014343641 discloses methods for brain cooling. Such methods comprise cooling assemblies including elongate tubular members, a reservoir containing pressurized gas and a manifold connecting the reservoir and the elongate tubular members. The elongate tubular members are inserted only into the patient's nostrils and a pressurized gas is directly delivered onto the surface of the patient's nasal cavity through a plurality of ports passing only through the nasal cavity in the elongate tubular members. The pressurized gas is vaporized in one or two nasal cavities only and it is vented through the same pathway. The gas is inserted in a temperature of −20° C. The method described in said document has neither control of the gas temperature vaporized onto the nasal cavities nor continuous feedback control from the brain temperature. Also, the method described in said document promotes direct contact of the evaporated gas with nasal mucosae which may cause adverse events such as periorbital gas emphysema, coolant in facial sinus, epistaxis and tissue damage, as described by the authors. Additionally, the gas may be absorbed by lung circulation.

As described in US2014343641, the delivery of fluid causes cooling by direct heat transfer. Said direct heat transfer and the contact of the fluid to the nasal cavity can cause damage to the patient's nasal cavity.

Recent published review manuscripts²⁸⁻³⁰ have disclosed some possible modalities of therapeutic hypothermia, however, none had suggested any similar catheter as disclosed in the present invention.

Therefore, based on the searches, documents anticipating or suggesting the teaching of the present invention were not found, therefore, the solution herein proposed presents novelty and inventive activity in view of the state of the art.

Thus, the present invention presents a solution for the problem of temperature management without causing further problems to the patient which undergoes treatment.

To date, none of the nasopharyngeal methods was able to achieve the exclusive brain cooling. Despite its effectiveness in promoting reduction of brain temperature, some decrease in body temperature of up to 2.5 degrees Celsius¹⁵ are still observed, which can still cause deleterious systemic side effects. The known intranasal devices have not been designed for prolonged use and novel devices should address this gap.

### BRIEF DESCRIPTION OF THE INVENTION

The present invention aims to solve the problem presented in the state of the art with a new device, system and method which solves the problem of:
temperature management;
preventing or reducing secondary lesions to the nervous system;
preventing or reducing primary lesions to the nervous system caused by surgical or clinical interventions in which nervous system is brought to risk;
preventing, reducing or treating spontaneous bleeding or bleeding caused by surgeries;
preventing, reducing or treating lesions of body parts;
treating body parts with thermal cauterization
metabolism control;

The present invention promotes heat transfer using a catheter which enables wide contact of an expansible section with the outer conformation.

The present invention

a. promotes heat transfer using any kind of fluid.
b. promotes heat transfer using a catheter which may be inserted through one cavity and exteriorized through another cavity or inserted through one cavity and exteriorized through the same cavity.
c. promotes heat transfer using fluid in steady or moving state.
d. enables heat transfer to organs or cavities with therapeutic purposes also enabling exclusive brain cooling
e. promotes heat transfer instead of using effective counteracting measures to promote whole body temperature normalization
f. enables implementation of mucosal protection means during heat transfer, minimizing mucosal damage caused by the intervention.
g. provides improvement to the status quo of the present temperature management medical devices due to:
its ability of enabling effective heat transfer;
its ability to exclusively cool a certain body part.

The above advantages being able to be provided with a selective body part temperature control method, an increase in the temperature gradient between the target body part and the rest of the body part and the lessening of surgical complications by means of the non-invasive device of the present invention.

In a first aspect, the present invention defines a catheter comprising:
at least one expandable section comprising at least one opening;
wherein the wall of the expandable section is capable of adapting to the conformation of an outside structure by means of pressure of a fluid;
wherein the at least one opening is capable of receiving at least one fluid inlet means and capable of allowing said fluid inlet to return through the same opening.

In a second aspect, the present invention defines a system for target temperature management comprising:
at least one catheter as defined above; and
at least one fluid provider connected to the catheter;
wherein the expandable section of the catheter is in contact with or inside at least one matter to manage temperature of the matter.

In a third aspect, the present invention defines a method for target body temperature management comprising the steps of:
positioning the expandable section of the catheter as defined above in contact with or inside at least one body part; and
infusing fluid through the system as defined above;
wherein said temperature management is done by cooling or heating by means of infusing the fluid through the expandable section of the catheter;
wherein the expandable section of the catheter is expanded by the pressure or volume of the fluid enough to enable contact of its wall with the conformation of the body part.

These and further objects of the invention would be immediately appreciated by the person skilled in the art and by firms of the same segment, and will be described with enough details so as to allow its reproduction as follows.

### BRIEF DESCRIPTION OF THE DRAWINGS

In order to clearly define the content of the present application, the following drawings are presented:

FIG. 1 shows four examples of embodiments of the catheter (1) of the present invention.

FIG. 2 shows an embodiment of the catheter (1) wherein the expandable section (2) presents a single opening (3) (without segmentation (3.1)) and fluid provider (8) connected to the catheter (1).

FIG. 3 shows an embodiment of the catheter (1) with an expandable section (2) (with segmentation (3.1)) including different segmentation (3.1) sizes and fluid provider (8) connected to one or two of the catheter openings (3).

FIG. 4 shows an embodiment of the catheter (1) with two openings (3) (curved shape) and fluid provider (8) to one or two of the catheter openings (3).

FIG. 5 shows an embodiment of the catheter (1) with one expandable section (2) with two openings (3) (curved shape). Two non-expandable sections (4, 5) are connected to the expandable section (2). Further showing an example of use in esophageal position.

FIG. 6 shows an embodiment of the catheter (1) with one expandable section (2) with two openings (3) (straight shape) with different expandable section sizes.

FIG. 7 shows an embodiment of the catheter (1) with one expandable section (2) with two openings (3) (straight shape) positioned in a curved conformation.

FIG. 8 shows an embodiment of the catheter (1) with one expandable section (2) with two openings (3) (straight shape) illustrating at least one non-expandable section (4, 5) which may be connected to one or two of the openings (3) of the expandable section (2).

FIG. 9 shows an embodiment of the catheter (1) with one expandable section (2) with two openings (3) (straight shape positioned in a curved conformation) illustrating at least one non-expandable section (4, 5) which may be connected to one or two of the openings (3) of the expandable section (2).

FIG. 10 shows embodiments of the catheter (1) with one expandable section (2) with two openings (3) (straight shape) illustrating two non-expandable sections (4, 5) connected to the two of the openings (3) of the expandable section (2). Further illustrating three different expandable section sizes.

FIGS. 11, 12 and 13 show the embodiment of the VERSION 1 of the catheter of the present invention.

FIG. 11 shows an embodiment of the version 1 of the nasopharyngeal catheter (1). 2. Expandable section, 3. Opening as a connection of the distal end of a first non-expandable section (4) with the proximal end of an expandable section (2), 4. First non-expandable tubular section, 5. Second non-expandable tubular section, 6. Guidance means, 10. Connector (connects the proximal end of the first non-expandable tubular section with the system).

FIG. 12 shows an embodiment of the version 1 of the nasopharyngeal catheter detailed. 2. Expandable section, 4. First non-expandable tubular section, 5. Second non-expandable tubular section, 6. Guidance mean, 10. Connector (connects the proximal end of the first non-expandable tubular section with the system), 11. Second opening as a connection of the distal end of the expandable section with the proximal end of the second non-expandable tubular section.

FIG. 13 shows an embodiment of the version 1 of the nasopharyngeal catheter in detail.

FIGS. 14 to 17 show pictures of the VERSION 1.

FIG. 14 shows a picture of the embodiment of the VERSION 1 of the catheter without the connector, showing the 3 sections, the guidance mean.

FIG. 15 shows a picture of the embodiment of the version 1 of the nasopharyngeal catheter detailed. with the connector, showing the amplified visions of the connector with the guidance mean (6) inserted and the tip (6.2) of the guidance mean used in this version.

FIG. 16 shows a picture of the embodiment of the version 1 of the nasopharyngeal catheter detailed showing tip (6.2) of the guidance mean (6) used in this version the amplified.

FIG. 17 shows pictures of an embodiment of the version 1 of the nasopharyngeal catheter detailed showing tip (6.2) of the guidance mean (6) used in this version the amplified and the guidance mean detailed.

FIGS. 18 and 19, show pictures of the VERSION 1 filled with fluid and with the expansion of the expandable section.

FIG. 18 shows pictures of an embodiment of the version 1 of the nasopharyngeal catheter with other connection (white connector) with the proximal end of the first non-expandable tubular section. 2. Expandable tubular section, 4. First non-expandable tubular section, 5. Second non-expandable tubular section. The picture shows the catheter filled with fluid and connected to the closed loop system.

FIG. 19 shows pictures of an embodiment of the version 1 of the nasopharyngeal catheter. The pictures show the catheter filled with fluid and connected to the closed loop system. First picture at the start of the circulating system, the second picture, at full flow and maximum expansion (4 cm) and the third at the shutting down.

FIG. 20 shows dimensions of an embodiment of the version 1 of the catheter.

FIG. 21 shows an illustration showing the three sections of the present invention which shows the surface area in contact with the expandable section of the catheter. 2. Expandable tubular section, 4. First non-expandable tubular section, 5. Second non-expandable tubular section.

FIG. 22 shows an embodiment of the version 2 of the nasopharyngeal catheter with shorter expandable section.

FIG. 23 shows a picture of an embodiment of the version 2 of the nasopharyngeal catheter with shorter expandable section.

FIG. 24 shows a picture of an embodiment of the version 2 of the nasopharyngeal catheter inside a transparent hose showing the catheter expanding inside the hose and adapting to the inner surface area.

FIG. 25 shows a sequence of pictures describing an embodiment of the method for the use of the nasopharyngeal catheter singly in a closed loop, being able to exempt the use of the system.

FIG. 26 shows an embodiment of a version 3 of the nasopharyngeal catheter (1) with shorter expandable section. 2. Expandable section, 4. First non-expandable tubular section, 5. Second non-expandable tubular section, 6. Guidance mean, 7. Tubular port, 10. Connector (connects the proximal end of the first non-expandable tubular section with the system).

FIG. 27 shows an embodiment of the version 3 of the nasopharyngeal catheter detailed. 2. Expandable section, 4. First non-expandable tubular section, 5. Second non-expandable tubular section, 6. Guidance mean, 7.1. Port for syringe connection to fill the inflatable section with air, 7.2. Second Tubular connection of the port with the inflatable section, 7.3. Inflatable section with anchoring purposes, 7.4 First Tubular connection of the port with the inflatable section, 10. Connector (connects the proximal end of the first non-expandable tubular section with the system).

FIG. 28 shows an embodiment of the version 3 of the catheter in detail.

FIG. 29 shows an embodiment of a version 4 of the nasopharyngeal catheter (1) with shorter expandable section. 2. Shorter expandable section, 4. First non-expandable tubular section, 5. Second non-expandable tubular section, 6. Guidance mean, 7. Tubular ports, 10. Connector (connects the proximal end of the first non-expandable tubular section with the system).

FIG. 30 shows an embodiment of the version 4 of the nasopharyngeal catheter detailed. 2. Expandable section, 4. First non-expandable tubular section, 5. Second non-expandable tubular section, 6. Guidance mean, 7.1. Port for syringe connection to fill the inflatable section with air, 7.2 Tubular connections of the ports to the inflatable section and protection, 7.3. Inflatable section with anchoring purposes, 7.4. First Tubular connection of the port with the inflatable section, 7.5 Port for syringe connection to apply mucosal protective agents (like gels and oils), 10. Connector (connects the proximal end of the first non-expandable tubular section with the system).

FIG. 31 shows an embodiment of the version 4 of the catheter in detail.

FIG. 32 shows a graphic with the core temperatures (rectal, lung and esophageal artery) and the cerebral hemispheres temperatures. *Time at which the difference between the brain and systemic temperatures reach statistical significance (p<0.001). Values expressed as mean and standard deviation.

FIG. 33 illustrates mean temperature difference between brain and core.

FIG. 34 illustrates the cooling rate of the left brain hemisphere. Brain cooling was more intense during the first twenty minutes.

FIG. 35 shows a graphic with the systemic hemodynamic parameters (HR, MAP, CO) during hypothermia using the invention presented here.

FIG. 36 shows a graphic with the encephalic hemodynamic parameters during hypothermia using the invention presented here.

FIG. 37 shows a graphic demonstrating the superiority of the present invention over similar technologies with the purpose of nasopharyngeal cooling.

### DETAILED DESCRIPTION OF THE INVENTION

The term/expressions below are defined in the context of the present invention:

Catheter: are medical devices made of flexible or rigid hollow tube inserted through body channels or natural cavities in order to allow the passage of fluids or to dilate the pathway.

Expandable: means something able to increase in size in any dimension, for example in length or in diameter.

Non-expandable: means something not able to increase in size in any dimension, for example in length or in diameter.

Heat exchange/transfer is defined as the exchange of thermal energy between physical systems, depending on the temperature and pressure, by dissipating heat. The fundamental modes of heat transfer are conduction or diffusion, convection and radiation. Heat energy transferred between a surface and a moving fluid at different temperatures is known as convection.

Convective heat transfer may take the form of either forced or natural convection, wherein forced convection occurs when a fluid flow is induced by an external force, such as a pump, fan or a mixer, and natural convection is caused by buoyancy forces due to density differences caused by temperature variations in the fluid.

Typical convective heat transfer coefficient for some common fluids:
Forced Convection
   Air and gases: 10-1000 (W/(m²K))
   Water and liquids: 50-10000 (W/(m²K))
   Liquid metals: 5000-40000 (W/(m²K))
Natural Convection
   Air and gases: 0.5-1000 (W/(m²K))
   water and liquids: 50-3000 (W/(m²K))

Newton's Law of cooling succinctly describes conductive heat transfer:

Q=h·ΔT·A Equation 1.

In which:
Q=total conductive heat transfer
h=Convective Heat Transfer Coefficient
ΔT=temperature difference between the fluid and the surfaces involved in the transfer
A=Area of surfaces over which transfer is taking place

Temperature difference (ΔT) and area (A) are the most important influencers of heat transfer. Convective Heat Transfer Coefficient (h) is another key influencer in heat transfer of fluids. Convective Heat Transfer Coefficient (h) is dependent on the flow properties such as velocity, viscosity and type of media (gas or liquid), and other flow and temperature dependent properties. Velocity has the greatest affect with a direct relationship in convective heat transfer. Geometric shape of the surface and surface condition are other major influencers of heat transfer. Therefore, the more contact exists in between the surface and the fluid, the higher will be the heat exchange. Orientation to the flow may play a role as well.

Good heat exchange capacity: means a combination of features which will lead to fast and efficient heat exchange from the fluid to the surface.

Little heat exchange capacity: means a combination of features which will lead to slower and less efficient heat exchange from the fluid to the surface.

Matter: is defined as any substance or object which has a physical body. E.g. a cavity or a body part.

Protective means: refers to substances employed to protect the surface of the part that will be in contact with the catheter during heat transfer

Temperature management: targeted temperature management (TTM) is defined as an active treatment that tries to achieve and maintain a specific body temperature in a person for a specific duration of time in an effort to improve health outcomes¹.

### Catheter

In a first aspect of the present invention, the catheter comprises at least one expandable section comprising one opening, wherein the expandable section of the catheter is expanded by the pressure or volume of the fluid enough to enable contact of its wall with the conformation the body part.

The expandable section of the catheter was made with a thin wall with good heat exchange capacity. This section was responsible for the wide contact with the outside structure. This allows the catheter to provide an extensive surface area for heat exchange.

The expandable section of the catheter expanded inside the structure by the pressure generated by the circulating fluid.

Non-expandable sections, when present, were made with a wall presenting lower heat exchange capacity than the expandable section. This characteristic was implemented with the intention to avoid lesions of the outside structure caused by the temperatures of the fluids circulating inside the catheter.

On FIG. 1, a first embodiment of the present invention is illustrated.

On FIG. 25, a second embodiment of the inventions is illustrated. In this embodiment a syringe is used for the insertion of the fluid in the catheter, at a desired temperature and the system would be thereafter closed, maintaining the temperature.

### System for Target Temperature Management

In a second aspect, the present invention defines a system for target temperature management comprising at least one said catheter and means to provide a fluid to the catheter.

In an embodiment of the system, it comprises a heat exchanger, a circulating pump and connectors.

The heat exchanger can be a cooling or heater generator, wherein it may be coupled with a thermometer which allows an improved temperature management.

The pump generates a high flow of the fluid, which improves the heat exchange, and applies pressure inside the walls of the expandable section of the catheter. Said expandable section of the catheter is expanded by the pressure or volume of the fluid enough to enable contact of its wall with the conformation of the body part.

In one embodiment, the connectors consist of hoses which are coupled to the system using fast connectors.

In one embodiment, the system for target temperature management also comprises at least one mean to counteract the influence of the system throughout the remaining non-target structure. In one embodiment, the target structure is being cooled and the means for counteract are blankets, thermal blankets, thermal mattress, vascular catheters, thermal bags and combinations thereof.

### Method for Target Body Temperature Management

In a third aspect, the present invention defines a method for target body temperature management comprising the steps of:
positioning the expandable section of the catheter as defined above in contact with or inside at least one body part; and
infusing fluid through the system as defined above;
wherein said temperature management is done by cooling or heating by means of infusing the fluid through the expandable section of the catheter;
wherein the expandable section of the catheter is expanded by the pressure or volume of the fluid enough to enable contact of its wall with the conformation of the body part.

In an embodiment, the target body part for temperature management is the brain. In a further embodiment, the target body part is preferably one brain hemisphere.

In an embodiment, the method consists of the insertion of said catheter through a structure, wherein the catheter is connected to a heat exchange system of circulating fluid, using a continuous high flow pump which allows temperature management.

In one embodiment, the guidance means of the catheter presents a covered tip with rounded section which facilitates the insertion of the catheter through the body part.

In one embodiment, the method also comprises a continuous temperature measurement of the fluid through the whole system. In one embodiment, the measured temperature is a body part targeted by the system. Said continuous measurement allows tight control of the temperature based on the treatment strategies. In one embodiment, the temperature measurement of the body part targeted is done by using a thermometer, near infrared spectroscopy (NIRS), magnetic resonance imaging (MRI), radiometry or combinations thereof.

In one embodiment the temperatures measured consist of brain.

The present invention defines the following clauses:

Clause 1. Catheter (1) comprising:
at least one expandable section (2) comprising at least one opening (3);
wherein the wall of the expandable section (2) is capable of adapting to the conformation of an outside structure by means of pressure of a fluid;
wherein the at least one opening (3) is capable of receiving at least one fluid inlet means and capable of allowing said fluid inlet to return through the same opening (3).

Clause 2. Catheter (1) according to clause 1 wherein opening (3) contains a segmentation (3.1) in its area capable of directing the fluid inlet inwards and outwards.

Clause 3. Catheter (1) according to clause 2 wherein said segmentation (3.1) is directed towards the inside section of the expandable section (2).

Clause 4. Catheter according to clause 2 wherein said at least one opening (3) is continuously connected by means of fusion, screwing or interference connection to at least one non-expandable section (4).

Clause 5. Catheter (1) according to any one of clauses 1 to 4 comprising:
at least one first non-expandable tubular section (4); and
at least one expandable section (2);
wherein the expandable section (2) is connected to the non-expandable tubular section (4), said connection by the distal end (4.1) of the first non-expandable tubular section (4) to the proximal end (2.1) of the expandable section (2);
wherein the wall of the expandable section (2) is capable of adapting to the conformation of an outside structure by means of pressure of a fluid.

Clause 6. Catheter (1) according to any one of clauses 1 to 5 further comprising:
at least one second non-expandable section (5) connected to the expandable section.

Clause 7. Catheter (1) according to any one of clauses 1 to 6, further comprising at least one guidance means (6).

Clause 8. Catheter (1) according to any one of clauses 1 to 7, wherein the expandable section (2) is made of a material which has good heat exchange capacity.

Clause 9. Catheter (1) according to clause 7, wherein said guidance means (6) is a guidance wire which passes through at least one of the sections.

Clause 10. Catheter (1) according to clause 9 wherein the guidance wire (6.1) presents at least one covered tip (6.2).

Clause 11. Catheter (1) according to clause 10 wherein the covered tip (6.2) presents a rounded section.

Clause 12. Catheter (1) according to clause 1 wherein the material of the expandable section (2) has good heat exchange capacity.

Clause 13. Catheter (1) according to clause 12 wherein the material is a plastic polyurethane-based compound.

Clause 14. Catheter (1) according to clause 13 wherein the plastic polyurethane-based compound of the expandable section (2) is made of Neusoft 40A.

Clause 15. Catheter (1) according to clause 4 or 5 wherein the material of the non-expandable section (4) has little heat exchange capacity.

Clause 16. Catheter (1) according to clause 15 wherein the material is a plastic polyurethane-based compound.

Clause 17. Catheter (1) according clause 16 wherein the plastic polyurethane-based compound is Texin 50D.

Clause 18. Catheter (1) according to clause 9 wherein the guidance wire (6.1) is made of a material which is selected from the group consisting of SS pebax.

Clause 19. Catheter (1) according any one of clauses 1 to 18 further comprising:
at least one tubular port (7) which is located inside or adjacent to any section of the catheter (1) wall.

Clause 20. Catheter (1) according to clause 19 wherein the at least one tubular port (7) is infused with a protective means.

Clause 21. Catheter (1) according to clause 20 wherein said protective means is selected from the group consisting of an oil, gas, gel or combinations thereof.

Clause 22. System for target temperature management comprising:
at least one catheter (1) as defined in any one of clauses 1 to 21; and
at least one fluid provider (8) connected to the catheter (1);
wherein the expandable section (2) of the catheter (1) is in contact with or inside at least one matter to manage temperature of the matter.

Clause 23. System according to clause 22 wherein said matter is a body part including internal cavities, external cavities and solid organs.

Clause 24. System according to clause 23 wherein the body part is nasal cavities, nasopharynx, oropharynx, oral cavity, esophagus, stomach, small intestine, large intestine, rectus.

Clause 25. System according to clause 22 further comprising at least one mean for temperature management.

Clause 26. System according to clause 22 further comprising at least one means for counteracting (9) the cooling or heating of the rest of the body.

Clause 27. System according to clause 26 wherein said means for counteracting (9) the cooling or heating of the rest of the body is selected from the group consisting of blankets, thermal blankets, thermal mattress, vascular catheters, thermal bags and combinations thereof.

Clause 28. Method for target body temperature management comprising the steps of:
positioning the expandable section (2) of the catheter (1) as defined in any one of clauses 1 to 21 in contact with or inside at least one body part; and
infusing fluid through the system as defined in any one of clauses 22 to 27;
wherein said temperature management is done by cooling or heating by means of infusing the fluid through the expandable section (2) of the catheter (1);
wherein the expandable section (2) of the catheter (1) is expanded by the pressure or volume of the fluid enough to enable contact of its wall with the conformation the body part.

Clause 29. Method according to clause 28 wherein said contact is a substantial contact.

Clause 30. Method according to clause 28 further comprising:
when said temperature management is done by cooling to at least one body part, a heating means (9) for counteracting the cooling to the rest of the body will be applied; or
when said temperature management is done by heating to at least one body part, a cooling means (9) for counteracting the heating to the rest of the body will be applied.

Clause 31. Method according to any one of clauses 28 to 30 wherein it is for preventing or reducing secondary lesions to the nervous system.

Clause 32. Method according to any one of clauses 28 to 30 wherein it is for preventing or reducing primary lesions to the nervous system caused by surgical or clinical interventions in which the nervous system is brought to risk.

Clause 33. Method according to any one of clauses 28 to 30 wherein it is for preventing, reducing or treating spontaneous bleeding or bleeding caused by surgeries.

Clause 34. Method according to any one of clauses 28 to 30 wherein it is for preventing, reducing or treating lesions of body parts.

Clause 35. Method according to any one of clauses 28 to 30 wherein it is for treating body parts with thermal cauterization.

Clause 36. Method according to any one of clauses 28 to 35 wherein said catheter (1) is inserted to the target body part by:
inserting at least one catheter (1) through at least one of the openings of the body;
passing through the cavity intended for temperature management;
coming out from the same opening or another opening of the body.

Clause 37. Method according to clause 36 wherein the catheter (1) is inserted through the nasal cavity and comes out through the oral cavity.

Clause 38. Method according to clause 28 wherein said body part is the nasopharynx.

The present invention shows many advantages such as: its easiness to access the target body part, its possibility of performing a selective fast temperature management, between further technical and economic advantages.

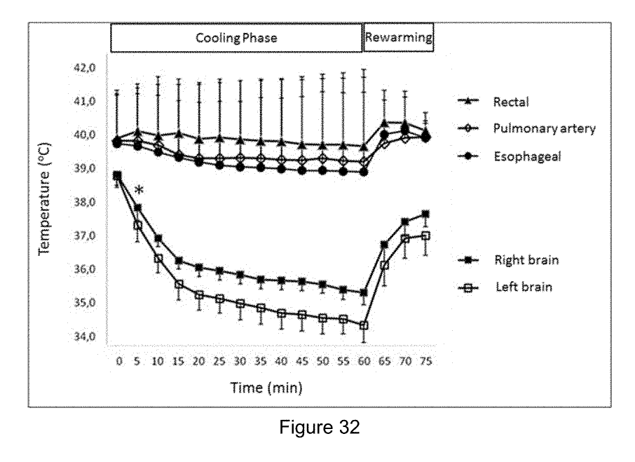

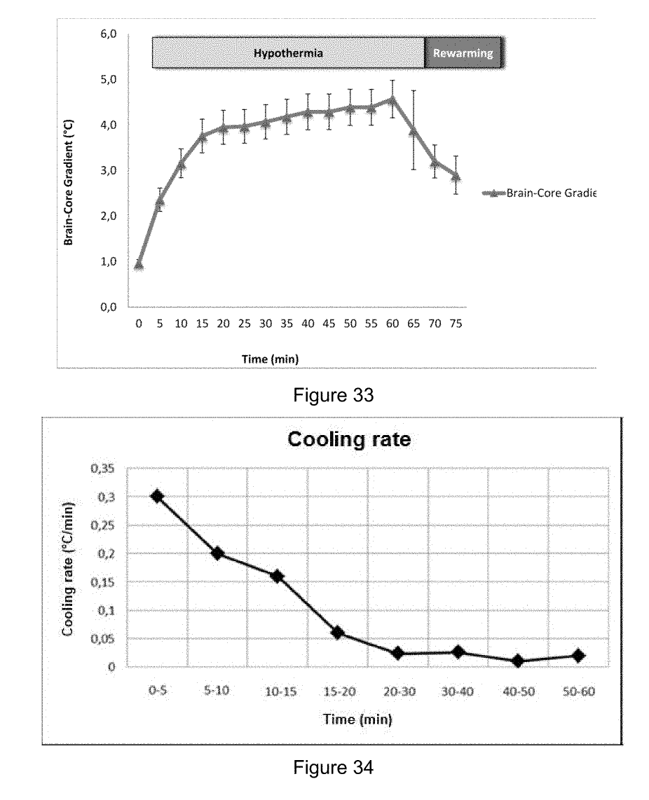

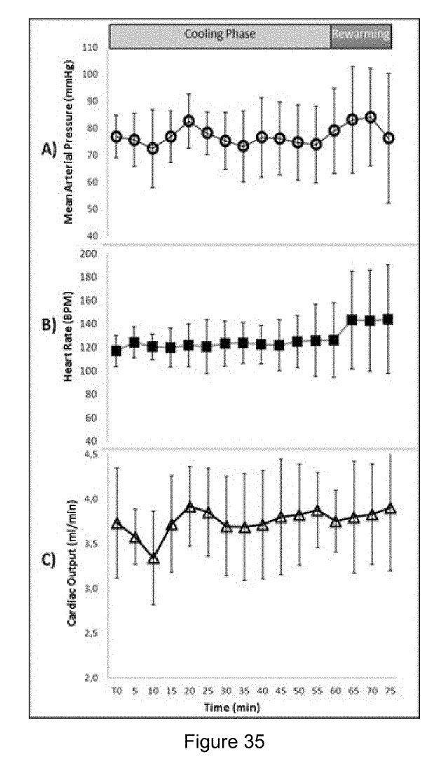

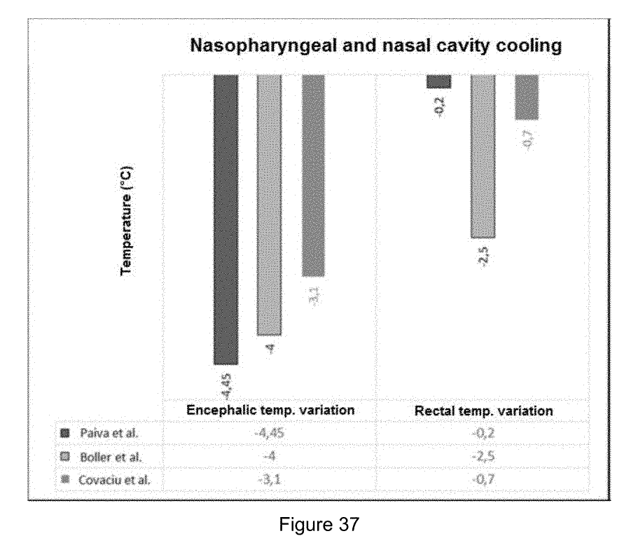

In one embodiment, the induction of exclusive brain hypothermia is feasible by means of a novel nasopharyngeal cooling device associated with body temperature preservation mechanisms. In one embodiment the brain temperature was lowered by 4.5° C., whereas the systemic temperatures remained stable (FIG. 32). The present invention shows superiority over the prior art as shown in FIG. 37.

The method implemented in our invention provides a substantial reduction of temperature of a target body part, in one embodiment the induction of exclusive brain hypothermia is feasible by the use of a nasopharyngeal cooling device associated with mechanisms of systemic temperature preservation. This exclusive brain cooling does not influence systemic and brain hemodynamics.

In one embodiment, the catheter is a nasopharynx cooling catheter that is used to decrease the temperature of encephalic tissue adjacent to the nasopharynx route. The components of the system facilitate catheter positioning. Inside the catheter, there will be continuous flow of liquid cooled by an external system connected to the catheter. The nasopharynx cooling catheter can be used on subjects with severe brain injuries which receive therapeutic brain cooling indication as method for neuroprotection. In one embodiment the system comprising the nasopharynx cooling catheter provides wider heat exchange surface between the catheter and the nasopharynx region which provide better results without complications as compared to the prior art.

In a preferred embodiment, the catheter of the present invention is associated with means for preserving systemic temperature such as blankets, thermal blankets, thermal mattress, vascular catheters, thermal bags and combinations thereof.

Earlier treatment with hypothermia induction was proven crucial to reduce motor and/or cognitive sequels for the patients and, in one embodiment, the catheter of the present invention allows to reduce the cerebral temperature in a fast and selective way, avoiding complication presented by other devices presented in the art. Preserving the systemic temperature and preserving the systemic and encephalic hemodynamics is also crucial to avoid further complications to patients undergoing hypothermia treatment.

### EXAMPLES

### Embodiments

### Example 1

Examples presented herein only illustrate one way of performing the invention, however, without restricting the scope of the same.

### Catheter Positioning

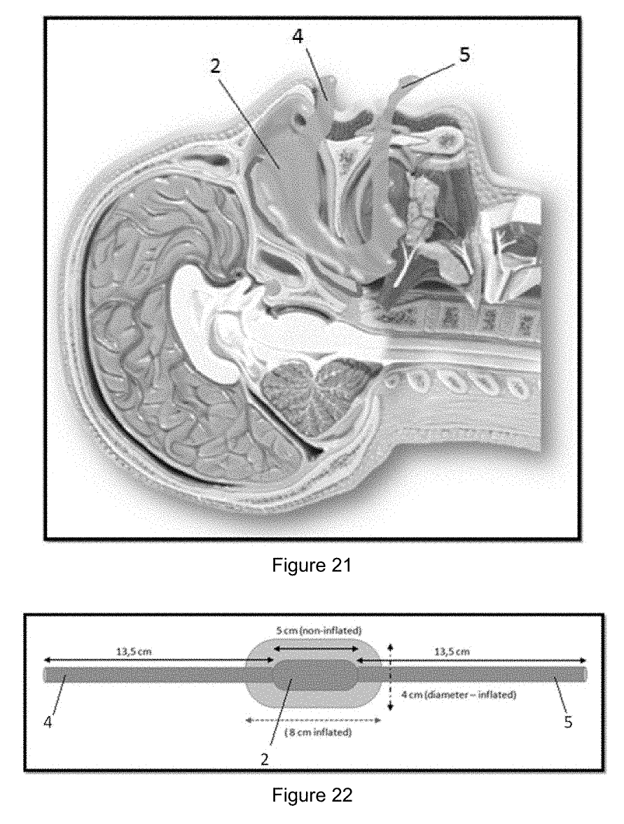

One example of the expandable section of the catheter positioning can be found in FIG. 21 which shows the expandable section taking the form of the nasopharynx region.

### Catheter Construction

One example of embodiment of the catheter presents a catheter comprising:
at least one expandable section (2) comprising at least one opening (3);
wherein the wall of the expandable section (2) is capable of adapting to the conformation of an outside structure by means of pressure of a fluid;
wherein the at least one opening (3) is capable of receiving at least one fluid inlet means and capable of allowing said fluid inlet to return through the same opening (3).

Further catheter examples are indicated in FIGS. 1 to 31 of the present application including also variants of said catheters.

FIG. 1 shows four variations of embodiments of the catheter presenting an expandable section (2) and one opening (3). Respectively, one present no segmentation, a first type of segmentation (3.1) which separates while maintaining sections adjacent to each other, a second type of segmentation (3.1) which more substantially separates the sections and a third type of segmentation (3.1) which separates even more substantially the sections.

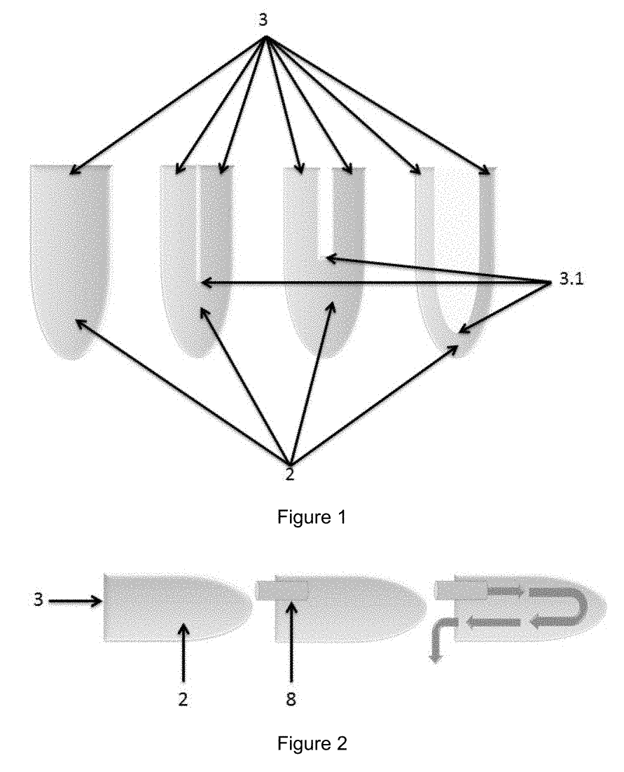

FIG. 2 shows a first variation of embodiment of the catheter presenting an expandable section (2) and one opening (3). One embodiment of connection to the fluid provider (8) is also represented.

FIG. 3 shows embodiments of a second variation of embodiment of the catheter presenting an expandable section (2), one opening (3) and a segmentation (3.1). Embodiments of connections to the fluid provider (8) are also represented.

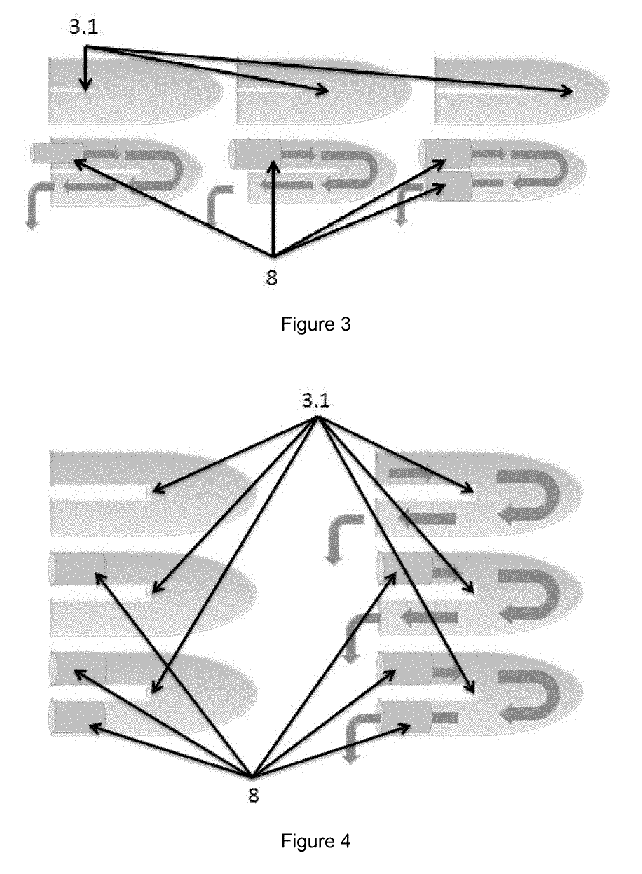

FIG. 4 shows embodiments of a third variation of embodiment of the catheter presenting an expandable section, one opening and a segmentation (3.1). Embodiments of connections to the fluid provider (8) are also represented.

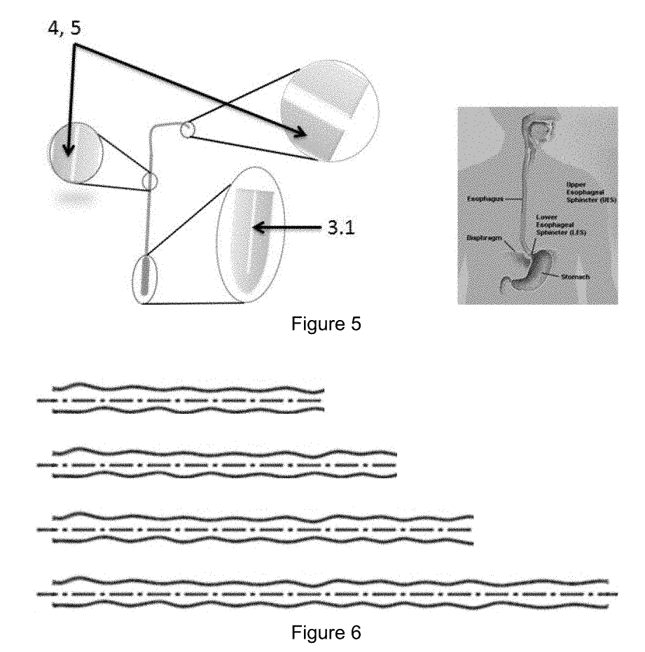

FIG. 5 shows an embodiment of the catheter working as an esophageal catheter.

FIG. 6 shows variations of embodiment of the expandable section of the catheter presenting openings in each of the opposite ends.

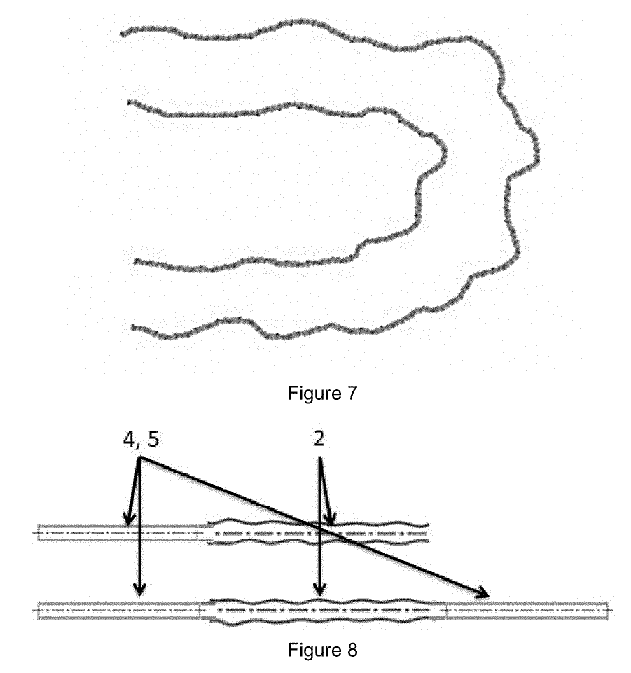

FIG. 7 shows an alternative configuration of the expandable section of the catheter.

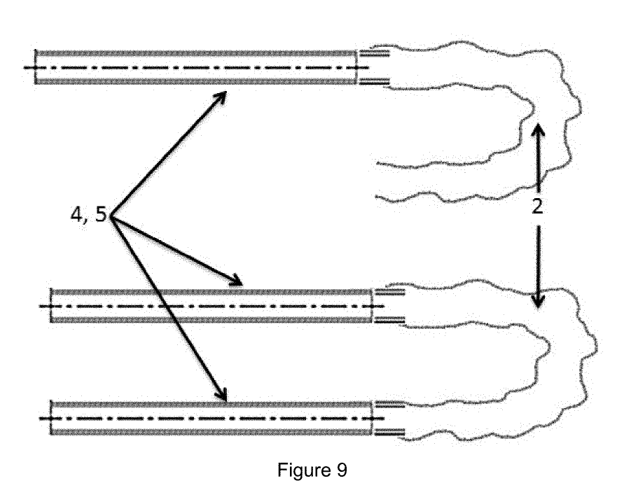

FIG. 8 shows embodiment of the catheter of the present invention presenting, respectively, a first non-expandable section (4) or a first (4) and a second (5) non-expandable section connected to ends of the expandable section (2).

FIG. 9 shows embodiment of the catheter of the present invention presenting, respectively, a first non-expandable section (4) or a first (4) and a second (5) non-expandable section connected to ends of the expandable section (2).

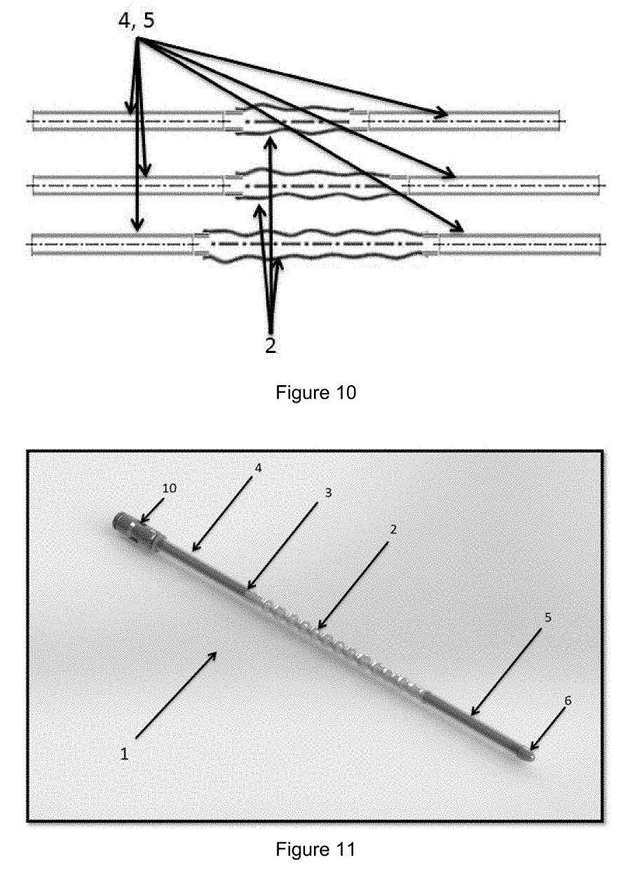

FIG. 10 shows variations of embodiments of the catheter of the present invention presenting, respectively, a first non-expandable section (4) or a first (4) and a second (5) non-expandable section connected to ends of the expandable section (2).

FIG. 11 shows an embodiment of the version 1 of the nasopharyngeal catheter (1). 2. Expandable section, 3. Opening as a connection of the distal end of a first non-expandable section (4) with the proximal end of an expandable section (2), 4. First non-expandable tubular section, 5. Second non-expandable tubular section, 6. Guidance means, 10. Connector (connects the proximal end of the first non-expandable tubular section with the system).

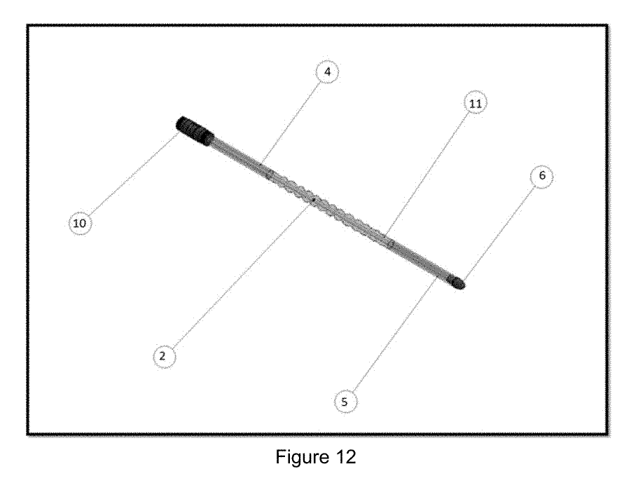

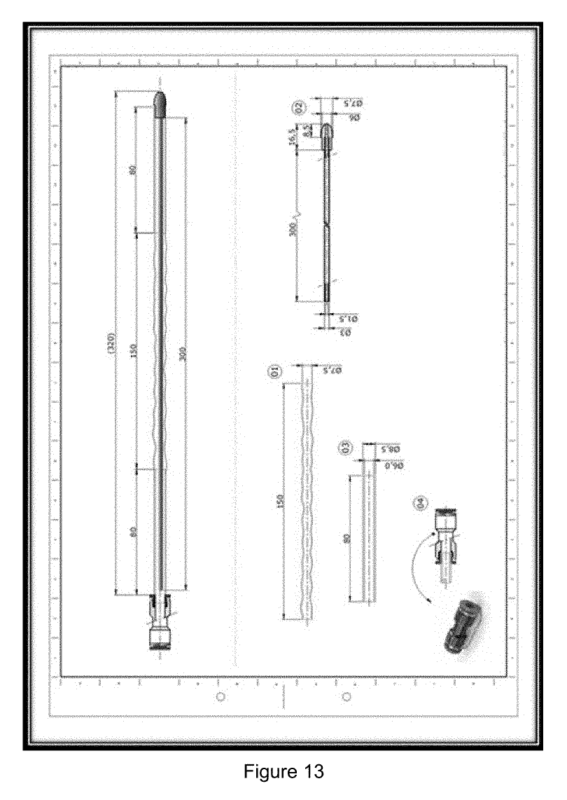

FIG. 12 shows an embodiment of the version 1 of the nasopharyngeal catheter detailed. 2. Expandable section, 4. First non-expandable tubular section, 5. Second non-expandable tubular section, 6. Guidance mean, 10. Connector (connects the proximal end of the first non-expandable tubular section with the fluid provider), 11. Second opening as a connection of the distal end of the expandable section with the proximal end of the second non-expandable tubular section.

The fluid provider (8) connected to the catheter by the proximal end of the first non-expandable tubular section provides fluid (in an embodiment said fluid is water) which passes through the first non-expandable tubular section (4), the expandable section (2) and the second non-expandable tubular section (5). Said expandable section (2) expands to the conformation of an external structure by the pressure of the fluid.

FIG. 13 shows an embodiment of the version 1 of the nasopharyngeal catheter in detail.

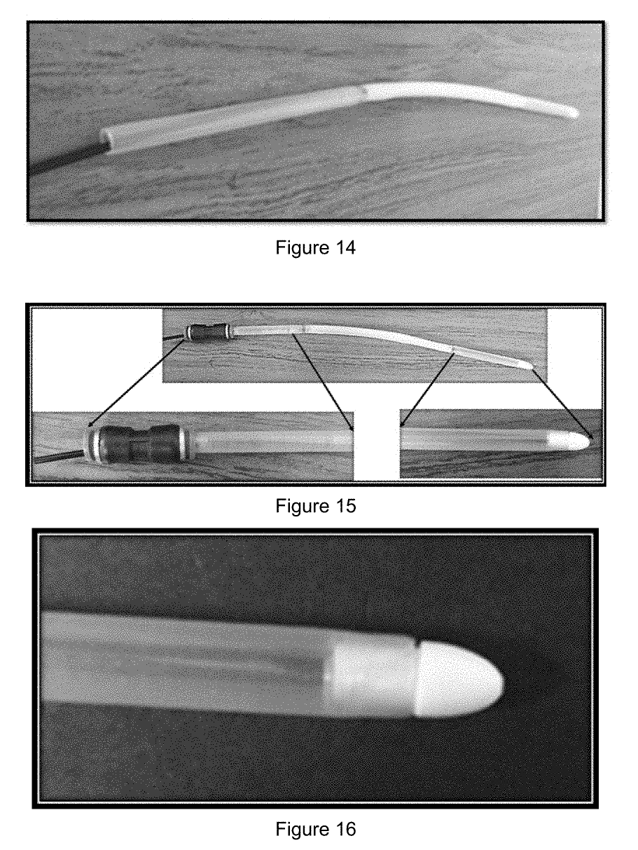

FIGS. 14 to 17 show pictures of the VERSION 1.

FIG. 14 shows a picture of the embodiment of the VERSION 1 of the catheter without the connector, showing the 3 sections, the guidance mean.

FIG. 15 shows a picture of the embodiment of the version 1 of the nasopharyngeal catheter detailed. with the connector, showing the amplified visions of the connector with the guidance mean (6) inserted and the tip (6.2) of the guidance mean used in this version.

FIG. 16 shows a picture of the embodiment of the version 1 of the nasopharyngeal catheter detailed showing tip (6.2) of the guidance mean (6) used in this version the amplified.

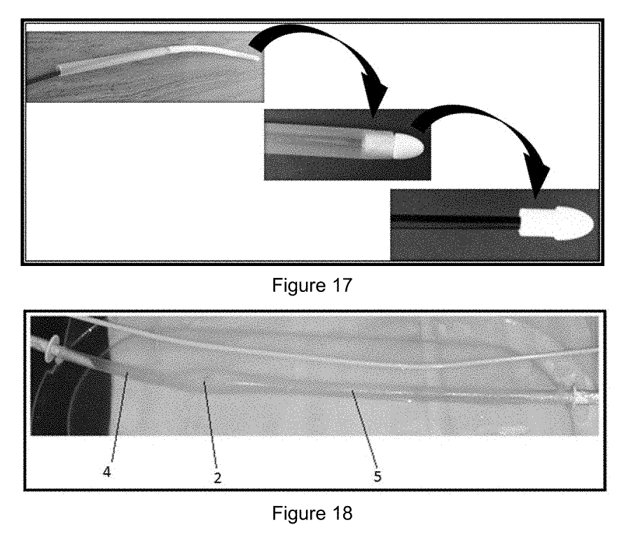

FIG. 17 shows pictures of an embodiment of the version 1 of the nasopharyngeal catheter detailed showing tip (6.2) of the guidance mean (6) used in this version the amplified and the guidance mean detailed.

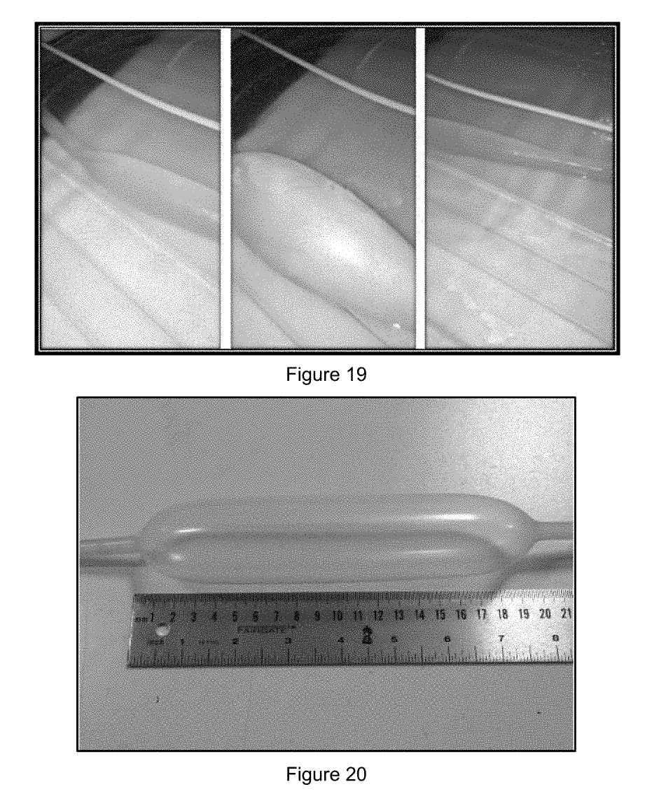

FIGS. 18 and 19, show pictures of the VERSION 1 filled with fluid and with the expansion of the expandable section.

FIG. 18 shows pictures of an embodiment of the version 1 of the nasopharyngeal catheter with other connection (white connector) with the proximal end of the first non-expandable tubular section. 2. Expandable tubular section, 4. First non-expandable tubular section, 5. Second non-expandable tubular section. The picture shows the catheter filled with fluid and connected to the closed loop system.

FIG. 19 shows pictures of an embodiment of the version 1 of the nasopharyngeal catheter. The pictures show the catheter filled with fluid and connected to the closed loop system. First picture at the start of the circulating system, the second picture, at full flow and maximum expansion (4 cm) and the third at the shutting down.

FIG. 20 shows dimensions of an embodiment of the version 1 of the catheter.

FIG. 21 shows an illustration showing the three sections of the present invention which shows the surface area in contact with the expandable section of the catheter. 2. Expandable tubular section, 4. First non-expandable tubular section, 5. Second non-expandable tubular section.

FIG. 22 shows an embodiment of the version 2 of the nasopharyngeal catheter with shorter expandable section.

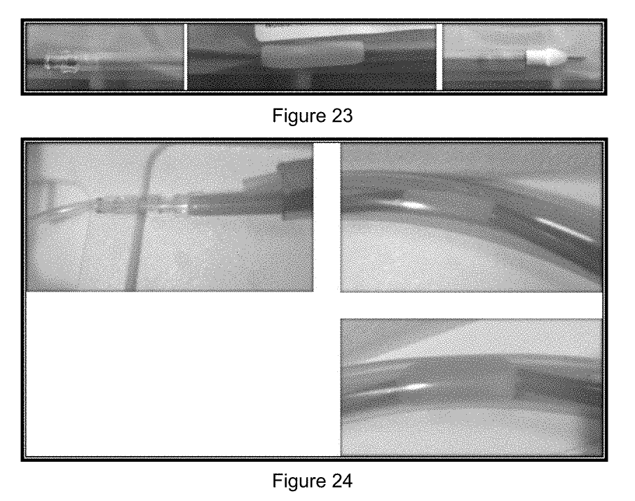

FIG. 23 shows a picture of an embodiment of the version 2 of the nasopharyngeal catheter with shorter expandable section.

FIG. 24 shows a picture of an embodiment of the version 2 of the nasopharyngeal catheter inside a transparent hose showing the catheter expanding inside the hose and adapting to the inner surface area.

FIG. 25 shows a sequence of pictures describing an embodiment of the method for the use of the nasopharyngeal catheter singly in a closed loop, being able to exempt the use of the system.

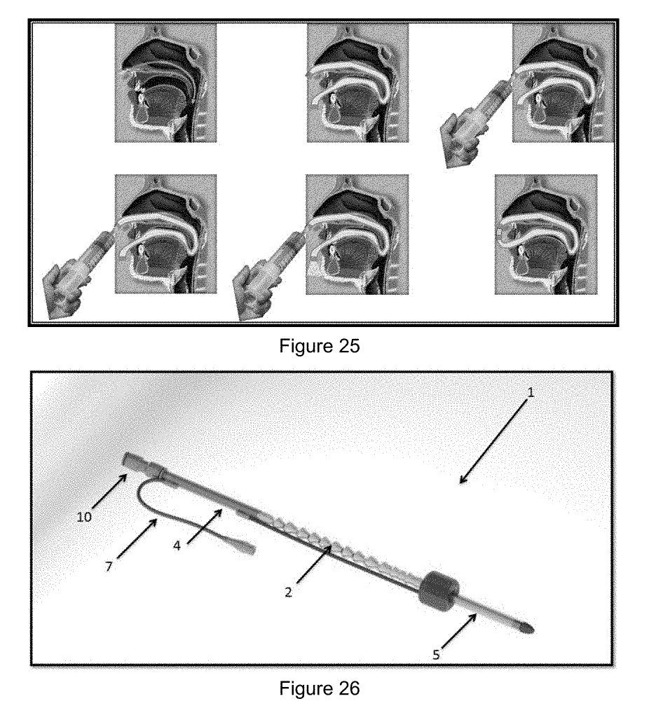

FIG. 26 shows an embodiment of a version 3 of the nasopharyngeal catheter (1) with shorter expandable section. 2. Expandable section, 4. First non-expandable tubular section, 5. Second non-expandable tubular section, 6. Guidance mean, 7. Tubular port, 10. Connector (connects the proximal end of the first non-expandable tubular section with the system).

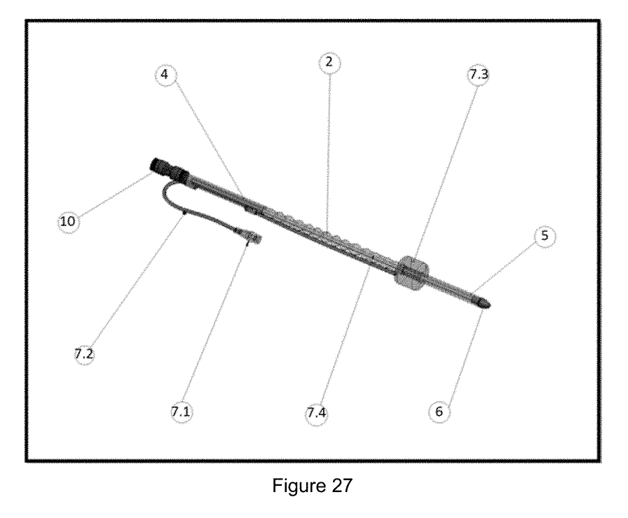

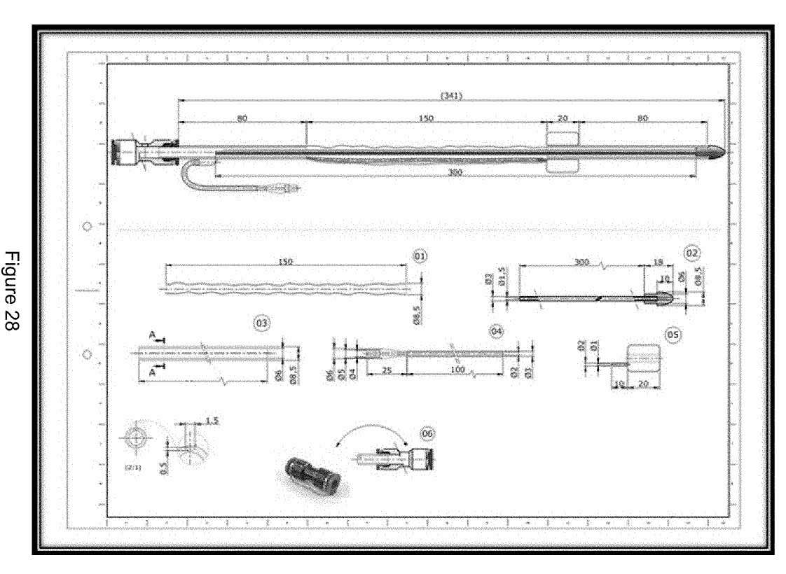

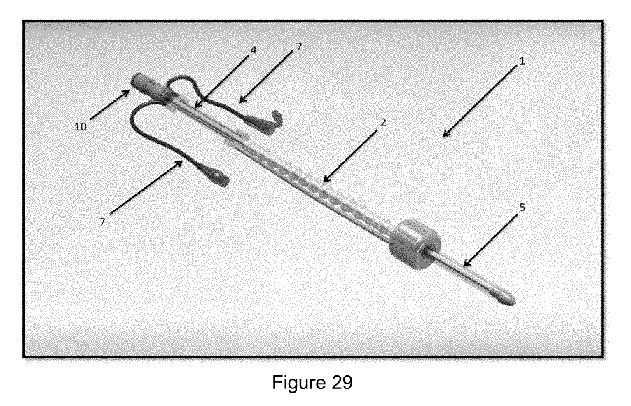

FIG. 27 shows an embodiment of the version 3 of the nasopharyngeal catheter detailed. 2. Expandable section, 4. First non-expandable tubular section, 5. Second non-expandable tubular section, 6. Guidance mean, 7.1. Port for syringe connection to fill the inflatable section with air, 7.2. Second Tubular connection of the port with the inflatable section, 7.3. Inflatable section with anchoring purposes, 7.4 First Tubular connection of the port with the inflatable section, 10. Connector (connects the proximal end of the first non-expandable tubular section with the system).

FIG. 28 shows an embodiment of the version 3 of the catheter in detail.

FIG. 29 shows an embodiment of a version 4 of the nasopharyngeal catheter (1) with shorter expandable section. 2. Shorter expandable section, 4. First non-expandable tubular section, 5. Second non-expandable tubular section, 6. Guidance mean, 7. Tubular ports, 10. Connector (connects the proximal end of the first non-expandable tubular section with the system).

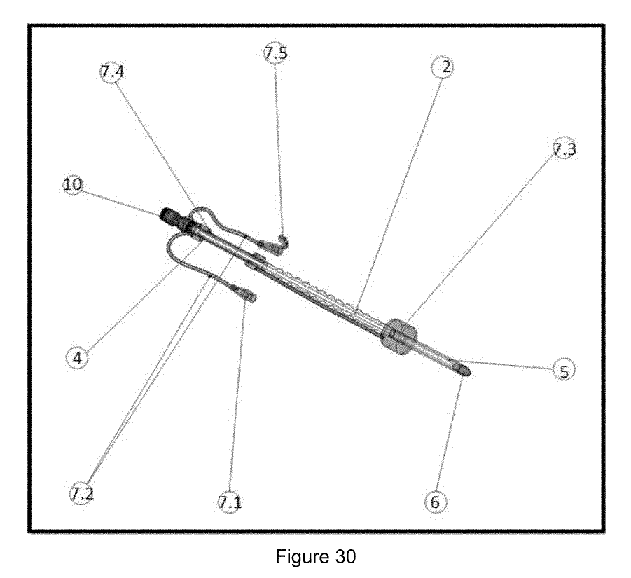

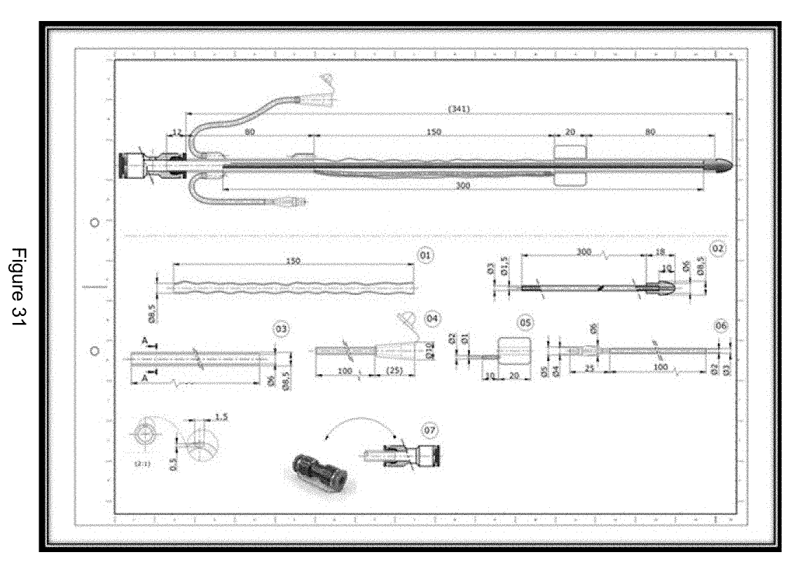

FIG. 30 shows an embodiment of the version 4 of the nasopharyngeal catheter detailed. 2. Expandable section, 4. First non-expandable tubular section, 5. Second non-expandable tubular section, 6. Guidance mean, 7.1. Port for syringe connection to fill the inflatable section with air, 7.2 Tubular connections of the ports to the inflatable section and protection, 7.3. Inflatable section with anchoring purposes, 7.4. First Tubular connection of the port with the inflatable section, 7.5 Port for syringe connection to apply mucosal protective agents (like gels and oils), 10. Connector (connects the proximal end of the first non-expandable tubular section with the system).

FIG. 31 shows an embodiment of the version 4 of the catheter in detail.

### Effectiveness Tests

### Selective Encephalic Hypothermia Induction

Nasopharyngeal cooling device implemented on the effectiveness test:
a. The nasopharyngeal cooling device was composed by a catheter, a heat exchanger, a circulating pump, its connectors and a thermometer. All components worked together in order to delivering controlled cooling to the nasopharynx using water for this purpose.
   i. Catheter was composed by one expandable section and two non-expandable sections.
      The expandable section was made with a thin wall with good heat exchange capacity. This section was responsible for the wide contact with the nasopharyngeal irregular conformation. This allowed the catheter to provide an extensive surface area for heat exchange.
      The two non-expandable sections were made with a thick wall which had lower heat exchange capacity than the expandable section. This characteristic was implemented with the intention to avoid nasal or nasopharyngeal thermal lesions caused by the low temperatures of the fluids circulating inside the catheter.
   ii. The catheter was inserted through the left nostril of all animals and visualized in the oral cavity using direct laryngoscopy. The catheter was connected to the heat exchange system of circulating cold water, between 0° C. and 2° C. using a continuous high flow generated by a water pump.
   iii. The expandable section of the catheter expanded inside the nasopharyngeal cavity due to the pressure generated by the circulating fluid pumped by the water pump.
b. The system consisted of a cooling (or heater) generator and a water pump.
   i. The cooling (or heater) generator was coupled to the parenchymal brain thermometer which allowed a continuous feedback to the temperature control during the experiment.
   ii. The water pump generated a high flow of the fluid in a closed loop layout.
c. The connectors consisted of hoses which were coupled to the system using fast connectors

Animals were subjected to anesthesia procedures, systemic and brain physiological monitoring, selective brain hypothermia via nasopharyngeal cooling, rewarming and euthanasia.

For the experiment, 10 (ten) healthy crossbred pigs (Landrace, Duroc and Pietrain), males and females, weighing 18 to 25 kg (mean, 20.6 kg±1.8) were used. They were purchased from breeders near São Paulo, within the legal procedures of the Department of Agriculture and Supply and Agricultural Defense and subsequently, were delivered at the School of Veterinary Medicine and Animal Science at the Universidade de São Paulo (USP), in a way that they could be sent to the School of Medicine at USP, on the morning of the trial.

Cooling was initiated 30 minutes after stabilization of respiratory and systemic hemodynamic variables. The cooling process lasted 60 minutes. After turning off the cooling system, brain rewarming occurred passively over 15 minutes.

During the experiment, body temperature of the animals was maintained by means of thermal mattresses and blankets. Body heating and nasopharyngeal cooling systems were turned on together and the first was maintained until the end of the rewarming phase.

The temperatures of both cerebral hemispheres were measured catheters inserted through cranial holes performed 0.5 cm lateral to the midline and 1 cm above the coronal suture bilaterally. Its extremities were positioned 1.5 cm from the cortical surface; the positioning of the distal end of the catheter was confirmed by intraoperative ultrasonography.

Systemic temperatures were measured by a thermometer placed in the lumen of the rectum (RT), another in the lumen of the esophagus (ET) and a third at the tip of a catheter (93A-131H-7F, Baxter Edwards Critical Care, Irvine, Calif., USA) placed in the lumen of the pulmonary artery (AT). These measurements were transmitted and stored on a multiparametric monitor (DX 2020, Philips/Dixtal, Manaus, AM, Brazil).

Systemic hemodynamic monitoring consisted in the measurement of the following physiological parameters: heart rate (HR), blood pressure (BP) and cardiac output (CO). HR was measured by three electrodes placed in the chest area, mean arterial pressure (MAP) via a polyethylene catheter (PE240) implanted in the lumen of the femoral artery and CO with a catheter (7F93A131H, Baxter Edwards Critical Care of Irvine, Calif., USA) positioned in the lumen of the pulmonary artery, using the intermittent thermo dilution technique 191.192. The catheter used for the measurement of CO was also used to measure the AT. Systemic hemodynamic data were captured and stored in a multiparameter monitor (DX 2020, Philips/Dixtal, Manaus, Brazil).

Through a system of multi-frequency ultrasound transducer coupled to a 4 to 8 MHz (MicroMaxx® SonoSite, Bothell, Wash., USA), cerebral blood flow velocity (CBFv) was measured in a porcine cerebral artery. Mean velocities, peak systolic velocity (SV), the end-diastolic velocities (EDV) and resistance index (RI) were recorded every five minutes.

Results obtained at the study performed with the present invention. Baseline temperatures

Baseline temperatures of the right and left cerebral hemispheres, had no significant differences (TCe0: 38.78±1.16° C.; TCd0: 38.82±1.12° C., p=0.7). Baseline esophageal (ET), rectal (RT:) and pulmonary artery temperatures (AT), also had no significant differences (ET: 39.74±0.86° C.; RT: 39.9±0.86° C., AT: 39.82±0.86° C., for relations between ET: RT, p=0.164; ET: AT, p=0.45; RT: AT, p=0.62).

### Temperature Measurement During Hypothermia

### Brain Temperature

In the nasopharyngeal cooling phase, there was a significant decrease in the temperature of both brain hemispheres (FIG. 32).

The temperature of the left hemisphere was reduced by 1.47±0.86° C. (95% CI: −2.09 to −0.85, p=0.0004) after 5 minutes and 2.45±1 02° C. (95% CI: −3.19 to −1.71, p<0.0001) after 10 minutes, reaching 4.45±1.36° C. (95% CI: −5.43 to −3.47, p<0.0001) after 60 minutes (FIG. 32 and Table 1).

The temperature of the right hemisphere (RbT) was also reduced, but to a lesser extent. After 60 minutes, the observed reduction was 3.52±0.94° C. (95% CI: −4.19 to −2.85, p<0.0001).

The temperature difference between the hemispheres was significant after twenty minutes of cooling (LbT: 35.24±1.49° C., RbT: 36.06° C.±0.98, p=0.014) and was maintained until the end of that phase.

There was a rate of temperature decrease in the left brain of 0.3° C./min in the first 5 minutes, and 0.16° C./min until 15 minutes. After this period, the rate was slower: 0.06 to 0.02° C./min until the end of the experiment.

TABLE 1
Variation of left brain temperature (LbT) compared to baseline
temperature. The LbT reduction became significant within the
first 5 minutes (p < 0.0004). Values expressed as mean.
Left brain Temperature (LbT)

| Time | Difference (compared | | |
|---|---|---|---|
| (min) | to baseline) | CI 95% | p |
| 5 | −1.47 | −2.09 a −0.85 | 0.0004 |
| 10 | −2.45 | −3.19 a −1.71 | <0.0001 |
| 15 | −3.21 | −4.14 a −2.28 | <0.0001 |
| 20 | −3.40 | −4.45 a −2.63 | <0.0001 |
| 25 | −3.65 | −4.51 a −2.79 | <0.0001 |
| 30 | −3.80 | −4.75 a −2.84 | <0.0001 |
| 35 | −3.93 | −4.86 a −2.30 | <0.0001 |
| 40 | −4.08 | −5.01 a −3.15 | <0.0001 |
| 45 | −4.13 | −5.06 a −3.20 | <0.0001 |
| 50 | −4.23 | −5.17 a −3.29 | <0.0001 |
| 55 | −4.25 | −5.17 a −3.33 | <0.0001 |
| 60 | −4.45 | −5.43 a −3.47 | <0.0001 |

### Core Temperature

Core temperatures showed no significant variation during the experiment as compared to baseline temperatures. The maximum reduction of the rectal temperature (RT)° C. was 0.23±1.16 (p=0.55), pulmonary artery (AT) was 0.62±0.95° C. (p=0.07) and esophageal temperature (ET) 0.84±1.93° C. (p=0.20).

### Difference Between Brain and Core Temperatures

There was a significant difference between the brain and core temperatures since the first 5 minutes of nasopharyngeal cooling and remained until the end of the procedure.

### Hemodynamic Monitoring

### Systemic Hemodynamic Measurements

The systemic hemodynamic parameters (HR, MAP, and CO) remained stable throughout the nasopharyngeal cooling phase. The HR remained at 117±23 bpm, and a MAP of 79.1±13.9 mmHg. There was a non-significant reduction of CO during the first 10 minutes (from 3.7±0.6 to 3.3±0.5 ml/min), which then stabilized. Tachycardia was observed during the rewarming phase.

### Brain Hemodynamic Measurements

There was no significant variation in CBF velocities during the nasopharyngeal cooling phase. After 60 minutes of cooling, a slight reduction of CBF velocities was noticed, along with increased resistance index.

FIG. 32 shows that during nasopharynx cooling, there was a significant reduction on the temperature of cerebral hemispheres while systemic temperature remained stable.

The temperature of left cerebral hemisphere reduced 1.47±0.86° C. after five minutes treatment, 2.45±1.02° C. after ten minutes treatment and 4.45±1.36° C. after sixty minutes treatment

There was significant difference between cerebral and systemic temperature since the first five minutes of nasopharynx cooling and the significant difference remained until the end of the hypothermia treatment as can be seen in FIG. 32.

### Systemic Hemodynamics

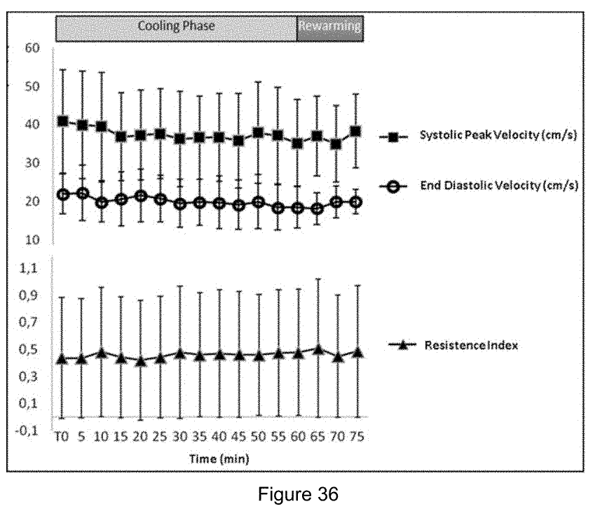

Systemic hemodynamic variables (FC—cardiac frequency, PAM—Mean Arterial Pressure and DC—Cardiac debt) remained stable during the hypothermia treatment. FC remained 117±23 bpm, PAM remained 79.1±13.9 mmHg and DC ranging from 3.3±0.5 mL/min to 3.7±0.6 mL/min during the first 10 minutes as can be seen in FIG. 36.

### Encephalic Hemodynamics

As can be seen in FIG. 36, no significative changes in the velocity of encephalic blood flow (FSE) were observed during hypothermia treatment.

So far, we have not found studies concerning the effects on systemic and brain physiological mechanisms resulting from isolated brain cooling. Similarly, the brain hemodynamics evaluated by Doppler ultrasonography had presented stable without significant changes in response to the exclusive hypothermia. The reduction of the CBF associated with systemic TH was previously demonstrated (33) and can be justified by the systemic hemodynamic variations secondary to whole body cooling.

The person skilled in the art will appreciate the knowledge presented herein and will be able to reproduce the embodiments of the invention and other variants, which are encompassed by annexed claims.

### REFERENCES

1. Peberdy M A, Callaway C W, Neumar R W, Geocadin R G, Zimmerman J L, Donnino M, et al. Part 9: Post-Cardiac Arrest Care: 2010 American Heart Association Guidelines for Cardiopulmonary Resuscitation and Emergency Cardiovascular Care. Circulation 2010; 122:S768-86.
2. Arrich J, Holzer M, Havel C, Müllner M, Herkner H. Hypothermia for neuroprotection in adults after cardiopulmonary resuscitation [Internet]. In: The Cochrane Collaboration, editor. Cochrane Database of Systematic Reviews. Chichester, UK: John Wiley & Sons, Ltd; 2012 [cited 2015 May 15]. Available from: http://doi.wiley.com/10.1002/14651858.CD004128.pub3
3. Yenari M A, Hemmen T M. Therapeutic Hypothermia for Brain lschemia: Where Have We Come and Where Do We Go? Stroke 2010; 41:S72-4.
4. Fox J L, Vu E N, Doyle-Waters M, Brubacher J R, Abu-Laban R, Hu Z. Prophylactic hypothermia for traumatic brain injury: a quantitative systematic review. CJEM 2010; 12:355-64.
5. Crossley S, Reid J, McLatchie R, Hayton J, Clark C, MacDougall M, et al. A systematic review of therapeutic hypothermia for adult patients following traumatic brain injury. Critical Care 2014; 18:R75.
6. Krieger D W, Yenari M A. Therapeutic Hypothermia for Acute Ischemic Stroke: What Do Laboratory Studies Teach Us? Stroke 2004; 35:1482-9.
7. Polderman K H. Application of therapeutic hypothermia in the ICU: opportunities and pitfalls of a promising treatment modality. Part 1: Indications and evidence. Intensive Care Med 2004; 30:556-75.
8. Clifton G L. Is keeping cool still hot? An update on hypothermia in brain injury. Curr Opin Crit Care 2004; 10:116-9.
9. Bernard S A, Jones B M, Home M K. Clinical trial of induced hypothermia in comatose survivors of out-of-hospital cardiac arrest. Ann Emerg Med 1997; 30:146-53.
10. Covaciu L, Allers M, Enblad P, Lunderquist A, Wieloch T, Rubertsson S. Intranasal selective brain cooling in pigs. Resuscitation 2008; 76:83-8.
11. Trübel H, Herman P, Kampmann C, Novotny E, Hyder F. [Selective pharyngeal brain cooling]. Biomed Tech (Berl) 2003; 48:298-300.
12. Hagioka S, Takeda Y, Takata K, Morita K. Nasopharyngeal cooling selectively and rapidly decreases brain temperature and attenuates neuronal damage, even if initiated at the onset of cardiopulmonary resuscitation in rats. Critical Care Medicine 2003; 31:2502-8.
13. Doll H, Truebel H, Kipfmueller F, Schaefer U, Neugebauer E A M, Wirth S, et al. Pharyngeal selective brain cooling improves neurofunctional and neurocognitive outcome after fluid percussion brain injury in rats. Journal of neurotrauma 2009; 26:235-42.
14. Yu T, Barbut D, Ristagno G, Cho J H, Sun S, Li Y, et al. Survival and neurological outcomes after nasopharyngeal cooling or peripheral vein cold saline infusion initiated during cardiopulmonary resuscitation in a porcine model of prolonged cardiac arrest*. Critical Care Medicine 2010; 38:916-21.
15. Boller M, Lampe J W, Katz J M, Barbut D, Becker L B. Feasibility of intra-arrest hypothermia induction: A novel nasopharyngeal approach achieves preferential brain cooling. Resuscitation 2010; 81:1025-30.
16. Harris B, Andrews P, Murray G, Forbes J, Moseley O. Systematic review of head cooling in adults after traumatic brain injury and stroke. Health Technol Assess [Internet] 2012 [cited 2015 May 15]; 16. Available from: http://www.journalslibrary.nihr.ac.uk/hta/volume-16/issue-45
17. Polderman K H, Rijnsburger E R, Peerdeman S M, Girbes A R J. Induction of hypothermia in patients with various types of neurologic injury with use of large volumes of ice-cold intravenous fluid. Crit Care Med 2005; 33:2744-51.
18. Tanaka M, Nagasaki G, Nishikawa T. Moderate hypothermia depresses arterial baroreflex control of heart rate during, and delays its recovery after, general anesthesia in humans. Anesthesiology 2001; 95:51-5.
19. Sabharwal R, Coote J H, Johns E J, Egginton S. Effect of hypothermia on baroreflex control of heart rate and renal sympathetic nerve activity in anaesthetized rats. J Physiol (Lond) 2004; 557:247-59.
20. Weisser J, Martin J, Bisping E, Maier L S, Beyersdorf F, Hasenfuss G, et al. Influence of mild hypothermia on myocardial contractility and circulatory function. Basic Res Cardiol 2001; 96:198-205.
21. Sessler D I. Mild perioperative hypothermia. N Engl J Med 1997; 336:1730-7.
22. Fischer U M, Cox C S Jr, Laine G A, Mehlhorn U, Allen S J. Mild hypothermia impairs left ventricular diastolic but not systolic function. J Invest Surg 2005; 18:291-6.
23. Polderman K H, van Zanten A R H, Girbes A R J. The importance of magnesium in critically ill patients: a role in mitigating neurological injury and in the prevention of vasospasms. Intensive Care Med 2003; 29:1202-3.
24. Soliman H M, Mercan D, Lobo S S M, Mélot C, Vincent J-L. Development of ionized hypomagnesemia is associated with higher mortality rates. Crit Care Med 2003; 31:1082-7.
25. Busch H J, Eichwede F, Föddisch M, Taccone F, Wöbker G, Schwab T, et al. Safety and feasibility of nasopharyngeal evaporative cooling in the emergency department setting in survivors of cardiac arrest. Resuscitation 2010; 81:943-9.
26. Poli S, Purrucker J, Priglinger M, Sykora M, Diedler J, Rupp A, et al. Safety Evaluation of Nasopharyngeal Cooling (RhinoChill®) in Stroke Patients: An Observational Study. Neurocritical Care 2014; 20:98-105.
27. Doll H, Maegele M, Bohl J, Störkel S, Kipfmueller F, Schaefer U, et al. Pharyngeal Selective Brain Cooling Is Associated with Reduced CNS Cortical Lesion after Experimental Traumatic Brain Injury in Rats. Journal of Neurotrauma 2010; 27:2245-54.
28. Christian E, Zada G, Sung G, Giannotta S L. A review of selective hypothermia in the management of traumatic brain injury. Neurosurgical focus 2008; 25:E9.
29. Guimarães R D L. Hipotermia terapêutica pós-reanimacção cardiorrespiratória: evidências e aspectos práticos. Rev Bras Ter Intensive 2009; 21:65-71.
30. Harris B, Andrews P, Murray G, Forbes J, Moseley O. Systematic review of head cooling in adults after traumatic brain injury and stroke. Health Technology Assessment [Internet] 2012 [cited 2016 Jan. 13]; 16. Available from: http://www.journalslibrary.nihr.ac.uk/hta/volume-16/issue-45.

1. A catheter comprising:
at least one expandable section comprising at least one opening;
wherein a wall of the expandable section is adaptable to a conformation of an outside structure by means of pressure of a fluid; and
wherein the at least one opening is structured to receive at least one fluid inlet means and to allow said fluid inlet to return through the same opening.

2. The catheter according to claim 1, wherein the at least one opening is continuously connected by means of fusion, screwing, or interference connection to at least one non-expandable section.

3. The catheter according to claim 1, wherein:
a first of the at least one first non-expandable section is a non-expandable tubular section;
the expandable section is connected to the non-expandable tubular section, said connection being between a distal end of the non-expandable tubular section and a proximal end of the expandable section; and
the wall of the expandable section is structured to adapt to a conformation of an outside structure by means of pressure of a fluid.

4. The catheter according to claim 3, further comprising:
a second of the at least one non-expandable section connected to the expandable section.

5. The catheter according to claim 1, further comprising at least one guidance means.

6. The catheter according to claim 1, further comprising:
at least one tubular port which is located inside or adjacent to any section of a catheter wall.

7. A system for target temperature management, comprising:
at least one catheter comprising at least one expandable section comprising at least one opening;
wherein a wall of the expandable section is adaptable to a conformation of an outside structure by means of pressure of a fluid; and
wherein the at least one opening is structured to receive at least one fluid inlet means and to allow said fluid inlet to return through the same opening; and
at least one fluid provider connected to the catheter;
wherein the expandable section of the catheter is in contact with or inside at least one matter to manage temperature of the matter.

8. The system according to claim 7, wherein the matter is a body part including internal cavities, external cavities, and solid organs.

9. The system according to claim 7, further comprising at least one means for temperature management.

10. The system according to claim 7, further comprising at least one means for counteracting the cooling or heating of the rest of the body.

11. A method for target body temperature management, comprising the steps of:
positioning an expandable section of a catheter (1) in contact with or inside at least one body part; and
infusing fluid through the system;
wherein the catheter comprises at least one expandable section comprising at least one opening;
wherein a wall of the expandable section is adaptable to a conformation of an outside structure by means of pressure of a fluid; and
wherein the at least one opening is structured to receive at least one fluid inlet means and to allow the fluid inlet to return through the same opening;
wherein said temperature management is done by cooling or heating by means of infusing the fluid through the expandable section of the catheter;
wherein the expandable section of the catheter is expanded by the pressure or volume of the fluid enough to enable contact of a wall of the catheter with a conformation of the outside structure.

12. The method according to claim 11, wherein the method prevents of reduces secondary lesions to a nervous system.

13. The method according to claim 11, wherein the catheter is inserted to a target body part by:
inserting the catheter through at least one opening of the body;
passing the catheter through a cavity intended for temperature management; and
passing the catheter out from the at least one opening of the body or another opening of the body.

14. The method according to claim 13, wherein the catheter is inserted through a nasal cavity of the body and out through an oral cavity of the body.

15. The method according to claim 11, wherein the body part is a nasopharynx of the body.
